(19) United States
(12) Patent Application Publication
(10) Pub. No.: US 2017/0311932 A1
(43) Pub. Date: Nov. 2, 2017

(54) DEPTH STOP DEVICE FOR USE WITH BIOPSY TARGETING ASSEMBLY

(71) Applicant: Devicor Medical Products, Inc., Cincinnati, OH (US)

(72) Inventor: Jordan Rebellino, Cincinnati, OH (US)

(21) Appl. No.: 15/499,950

(22) Filed: Apr. 28, 2017

Related U.S. Application Data
(60) Provisional application No. 62/329,299, filed on Apr. 29, 2016.

(51) Int. Cl. A61B 10/02

(52) U.S. Cl. CPC A61B 10/0233

(57) ABSTRACT

A device for use with a biopsy cannula includes a housing and a locking assembly. The housing defines a shaft aperture sized to receive and at least partially circumferentially encompass a biopsy cannula. The locking assembly defines a lock opening configured to receive the biopsy cannula. The locking assembly includes a pair of lock members extending inwardly within the lock opening. The pair of lock members are configured to bind against the biopsy cannula upon translation of the locking assembly relative to the lock opening to a locking position. The locking member and the housing are configured to restrict the depth of insertion of the biopsy cannula through the lock opening when the pair of lock members are bound against the biopsy cannula in the locking position.

The present application claims priority to U.S. Provisional Patent Application No. 62/329,299, entitled “Drawstring Z-Stop Device for Use with Biopsy Targeting Assembly,” filed Apr. 29, 2016, the disclosure of which is hereby incorporated by reference in its entirety.

### BACKGROUND

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, Positron Emission Mammography (“PEM” guidance), Breast-Specific Gamma Imaging (“BSGI”) guidance or otherwise.

The state of the art technology for conducting a breast biopsy is to use a vacuum-assisted breast biopsy device. A current textbook in this area is “Vacuum-Assisted Breast Biopsy with Mammotome®”, available Nov. 11, 2012, copyright 2013 by Devicor Medical Germany GmBh, published in Germany by Springer Medizin Verlag, Authors: Markus Hahn, Anne Tardivon and Jan Casselman, ISBN 978-3-642-34270-7.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000;

U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 1, 2012; U.S. Pat. No. 8,206,316, entitled “Tetherless Biopsy Device with Reusable Portion,” issued on Jun. 26, 2012; U.S. Pat. No. 8,241,226, entitled “Biopsy Device with Rotatable Tissue Sample Holder,” issued on Aug. 14, 2012; U.S. Pat. No. 8,251,916, entitled “Revolving Tissue Sample Holder for Biopsy Device,” issued Aug. 28, 2012; U.S. Pat. No. 8,454,531, entitled “Icon-Based User Interface on Biopsy System Control Module,” published May 21, 2009, issued on Jun. 4, 2013; U.S. Pat. No. 8,532,747, entitled “Biopsy Marker Delivery Device,” issued Sep. 10, 2013; U.S. Pat. No. 8,702,623, entitled “Biopsy Device with Discrete Tissue Chambers,” issued on Apr. 22, 2014; U.S. Pat. No. 8,764,680, entitled “Handheld Biopsy Device with Needle Firing,” issued on Jun. 11, 2014; U.S. Pat. No. 8,801,742, entitled “Needle Assembly and Blade Assembly for Biopsy Device,” issued Aug. 12, 2014; U.S. Pat. No. 8,858,465, entitled “Biopsy Device with Motorized Needle Firing,” issued Oct. 14, 2014; U.S. Pat. No. 8,938,285, entitled “Access Chamber and Markers for Biopsy Device,” issued Jan. 20, 2015; U.S. Pat. No. 9,095,326, entitled “Biopsy System with Vacuum Control Module,” issued Aug. 4, 2015 and U.S. Pat. No. 9,095,326, entitled “Biopsy System with Vacuum Control Module,” issued Aug. 4, 2015. The disclosure of each of the above-cited U.S. Patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006 and now abandoned; U.S. Pat. Pub. No. 2008/0214955, entitled “Presentation of Biopsy Sample by Biopsy Device,” published Sep. 4, 2008; U.S. Pat. Pub. No. 2009/0131821, entitled “Graphical User Interface For Biopsy System Control Module,” published May 21, 2009, now abandoned; U.S. Pat. Pub. No. 2010/0152610, entitled “Hand Actuated Tetherless Biopsy Device with Pistol Grip,” published Jun. 17, 2010, now abandoned; U.S. Pat. Pub. No. 2010/0160819, entitled “Biopsy Device with Central Thumbwheel,” published Jun. 24, 2010, now abandoned; U.S. Pat. Pub. No. 2013/0053724, entitled “Biopsy Device Tissue Sample Holder with Bulk Chamber and Pathology Chamber,” published Feb. 28, 2013, will issue on May 3, 2016 as U.S. Pat. No. 9,326,755; U.S. Pat. Pub. No. 2013/0144188, entitled “Biopsy Device With Slide-In Probe,” published Jun. 6, 2013; and U.S. Pat. Pub. No. 2013/0324882, entitled “Control for Biopsy Device,” published Dec. 5, 2013. The disclosure of each of the above-cited U.S. Patent Application Publications, U.S. Non-Provisional Patent Applications, and U.S. Provisional Patent Applications is incorporated by reference herein.

A merely exemplary localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

In U.S. Pat. No. 7,831,290, issued Oct. 20, 2010, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions.

A Z-stop or depth stop device may enhance accurate insertion, and prevent over-insertion or inadvertent retraction of a biopsy device targeting cannula/sleeve and obturator. In particular, a Z-stop may engage the localization fixture or cube at a distance from the patient set to restrict the depth of insertion of a biopsy device needle into a patient. Known Z-stop devices are disclosed in U.S. Pat. No. 7,507,210, entitled “Biopsy Cannula Adjustable Depth Stop,” issued Mar. 24, 2009, the disclosure of which has been previously incorporated by reference herein.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of a depth stop device for use in MRI guided breast biopsy procedures, the depth stop device issecured to a cannula;

FIG. 2 depicts another perspective view of the depth stop device of FIG. 1, with the cannula removed;

FIG. 3 depicts an exploded perspective view of the depth stop device of FIG. 1;

FIG. 4 depicts a perspective view of a distal member of a housing of the depth stop device of FIG. 1;

FIG. 5 depicts a perspective view of a proximal member of the housing of the depth stop device of FIG. 1;

FIG. 6 depicts a perspective view of a locking assembly of the depth stop device of FIG. 1;

FIG. 7 depicts another perspective view of the locking assembly of FIG. 6;

FIG. 8 depicts a front elevational view of the locking assembly of FIG. 6;

FIG. 9 depicts a front elevational view of the depth stop device of FIG. 1, with the distal member removed and the locking assembly in a locked configuration;

FIG. 10 depicts another front elevational view of the depth stop device of FIG. 1, with the distal member removed and the locking assembly in an unlocked configuration;

FIG. 11 depicts still another perspective view of the depth stop device of FIG. 1, with the depth stop device in an unlocked configuration; and

FIG. 12 depicts a top cross-sectional view of the locking assembly of FIG. 6, the cross-section taken along line 12-12 of FIG. 8.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

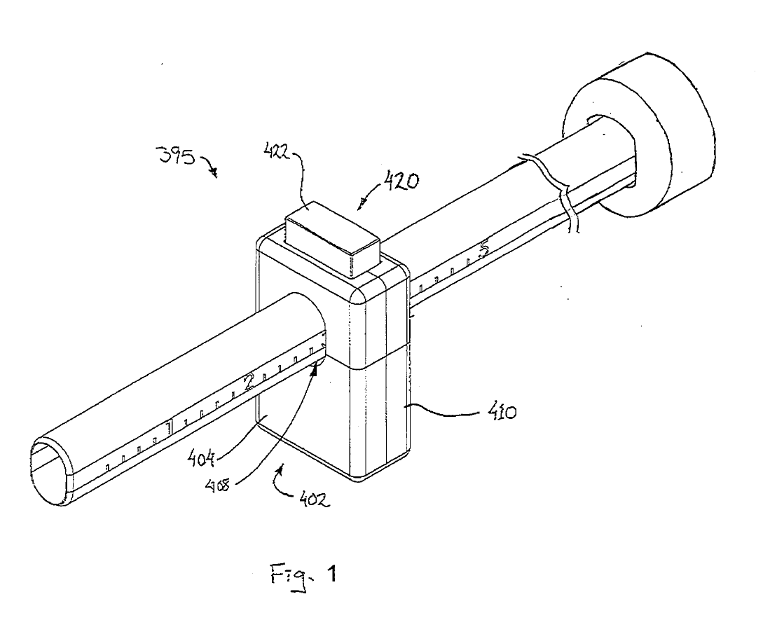

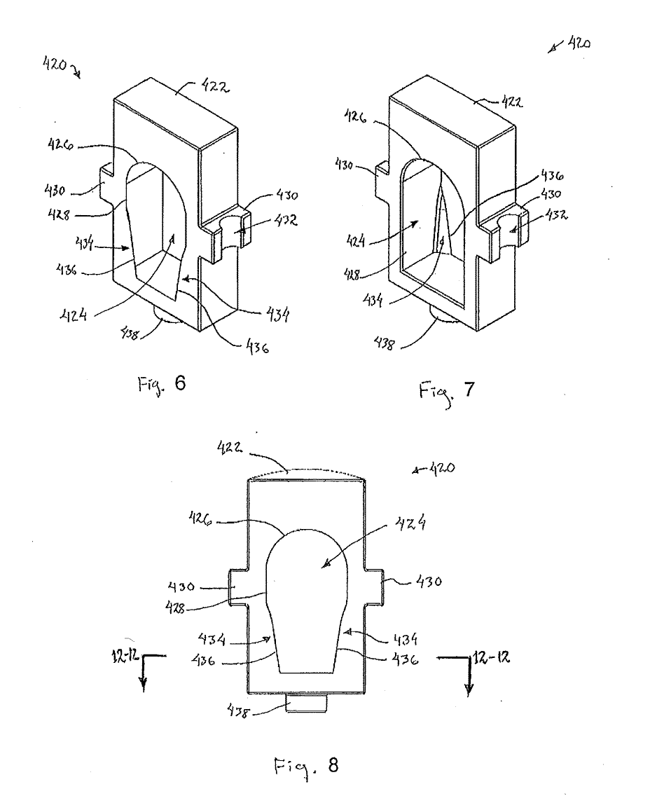

FIGS. 1, 2, 3, 4, 5, 6, 7, 8 and 9 of U.S. Pat. No. 7,507,210, incorporated by reference in its entirety, depict a perspective view of a biopsy system including a control module remotely coupled to a biopsy device, and including a localization fixture with a lateral grid plate used in conjunction with a rotatable cube to position an obturator or a probe of the biopsy device to a desired insertion depth as set by a ring stop.

During the breast biopsy procedure, typically the patient's breasts hang pendulously respectively into breast apertures on the examination table. For convenience, herein a convention is used for locating a suspicious lesion by Cartesian coordinates within breast tissue referenced to localization fixture and to thereafter selectively position an instrument, such as needle of probe that is engaged to holster portion to form biopsy device.

To enhance hands-off use of biopsy system, especially for repeated re-imaging within the narrow confines of a closed bore MRI machine, biopsy system may also guide obturator encompassed by cannula. Depth of insertion is controlled by a depth stop device longitudinally positioned on either needle or cannula. Alternatively, depth of insertion may be controlled in any other suitable fashion.

In typical MRI breast biopsy procedures, a targeting set comprising cannula and obturator is associated with probe. In particular, the obturator is slid into cannula and the combination is guided through guide cube to the biopsy site within the breast tissue. Obturator is then withdrawn from cannula, then the needle of the probe is inserted in cannula, and then biopsy device is operated to acquire one or more tissue samples from the breast via needle.

The amount of penetration within the breast is typically referred to as penetration along the Z-axis. Therefore, any mechanical means to control the depth of penetration along the Z-axis is typically referred to as a “Z-stop”. Alternatively, in some instances mechanical means to control the depth of penetration is referred to as a depth stop device. Throughout this patent application the mechanical means to control the depth of penetration along the Z-axis will be referred to as the Z-stop device, although it should be understood that the term “depth stop device” may also be used. In other words, the terms “Z-stop” and “depth stop device” should be read interchangeably.

During a MRI breast biopsy procedure a Z-stop device is affixed in some manner to the cannula, needle, probe or obturator to secure the probe's location within the breast. The Z-stop device may be of any suitable configuration that is operable to prevent cannula and obturator from being inserted further than desired. For instance, Z-stop device may be positioned on the exterior of cannula or needle, and may be configured to restrict the extent to which cannula or needle is inserted into a guide cube. It should be understood that such restriction by the Z-stop device may further provide a limit on the depth to which the combination of cannula and obturator or needle may be inserted into the patient's breast. Furthermore, it should be understood that such restriction may establish the depth within the patient's breast at which biopsy device acquires one or more tissue samples after obturator has been withdrawn from cannula and needle has been inserted in cannula. Examples of Z-stop devices that may be used with biopsy system are described in U.S. Pat. No. 8,568,333, entitled “Grid and Rotatable Cube Guide Localization Fixture for Biopsy Device,” issued Oct. 29, 2013, and incorporated by reference herein as mentioned previously.

In some circumstances a Z-stop device with a different mechanism of engaging the cannula may be desired. For instance, some Z-stop devices may be generally rotatable about cannula to lock and unlock depth stop device relative to cannula. However, in some instances, biopsy related equipment or imaging related equipment may limit access to depth stop device. It therefore may be difficult for an operator to fully grasp such a Z-stop device to make adjustments in penetration depth before or during a procedure. Accordingly, in some circumstances it may be desirable to provide a Z-stop device that can be actuated between a locked and unlocked state by other mechanisms or means. Various alternative Z-stop devices will be described in greater detail below; while other examples will be apparent to those of ordinary skill in the art according to the teachings herein. In particular, the Z-stop device examples described below may be used to assist in biopsy device needle targeting within a patient's breast using MRI guidance by permitting an operator to set a predetermined penetration depth. It should be understood that the Z-stop device examples discussed below may be readily used with many of the commercially available biopsy devices currently available on the market.

FIGS. 1-3 show an exemplary Z-stop device (395) that may be used with cannula, needle and/or obturator to provide guidance with respect to insertion depth as described above. For instance, depth of insertion of cannula or needle can be controlled by Z-stop device (395) being longitudinally positioned on cannula. Z-stop device (395) is sized to engage a guide cube and thereby restrict the depth of insertion of cannula relative to the guide cube. The guide cube is generally disposed within a grid plate or other support structure, which is secured to a patent table of an MRI imaging device to fix the guide cube relative to a patient. Thus, the cannula is prevented from being inserted beyond a specific depth relative to a patient via the guide cube once Z-stop device (395) abuts the guide cube. However, unlike some depth stop devices, Z-stop device (395) of the present example includes certain locking and actuation features to permit an operator to unlock and lock the Z-stop device (395) from a single contact point on Z-stop device (395).

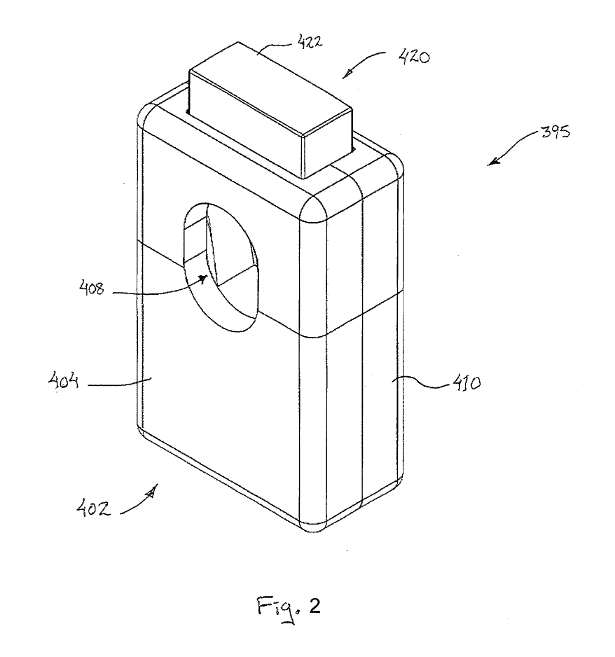

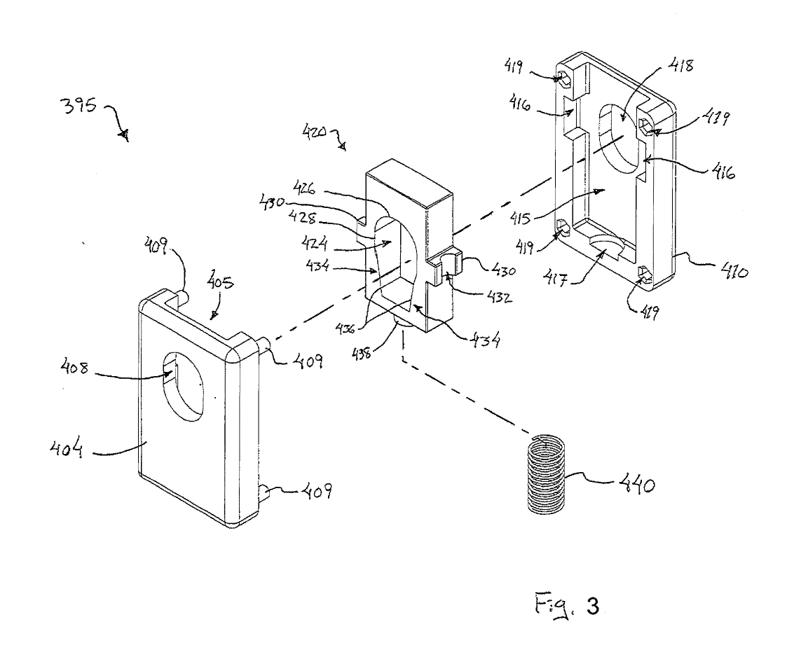

FIG. 1 shows Z-stop device (395) fixedly secured to the cannula described above. Although Z-stop device (395) is shown and described herein as being used in conjunction with cannula, it should be understood that in other examples Z-stop device (395) can be readily used with other components such as needle, obturator, or other similar surgical instruments. Z-stop device (395) is seen in greater detail in FIGS. 2 and 3. As can be seen, Z-stop device (395) comprises a housing (402) and a locking assembly (420) extending outwardly from housing (402).

In FIG. 3, the components disposed within housing (402) are visible. As will be discussed in further detail below, housing (402) at least partially encompasses locking assembly (420) to permit locking assembly (420) to be manipulated by a resilient member (440). Although resilient member (440) of the present example is shown as a coil spring, it should be understood that in other examples any other resilient feature may be used such as a leaf spring, or highly elastic materials comprising a variety of formations. As will be understood, Z-stop device (395) is generally configured to receive cannula or other similar devices such that cannula can be engaged by locking assembly (420).

In the present example housing (402) comprises a distal member (404) and a proximal member (410). As is best seen in FIG. 3, distal member (404) and proximal member (410) together form an interlocking longitudinally split configuration to thereby encompass at least a portion of locking assembly (420). Although distal member (404) and proximal member (410) are shown as forming a generally longitudinally split configuration, it should be understood that in some examples members (404, 410) are instead divided into top and bottom sections of housing (402). In such examples one member (404, 410) defines a top portion of housing (402), while another member (410, 404) defines a bottom portion of housing (402). Regardless of the particular configuration formed by members (404, 410), it should be understood that the components described herein with respect to each member (404, 410) are substantially similar.

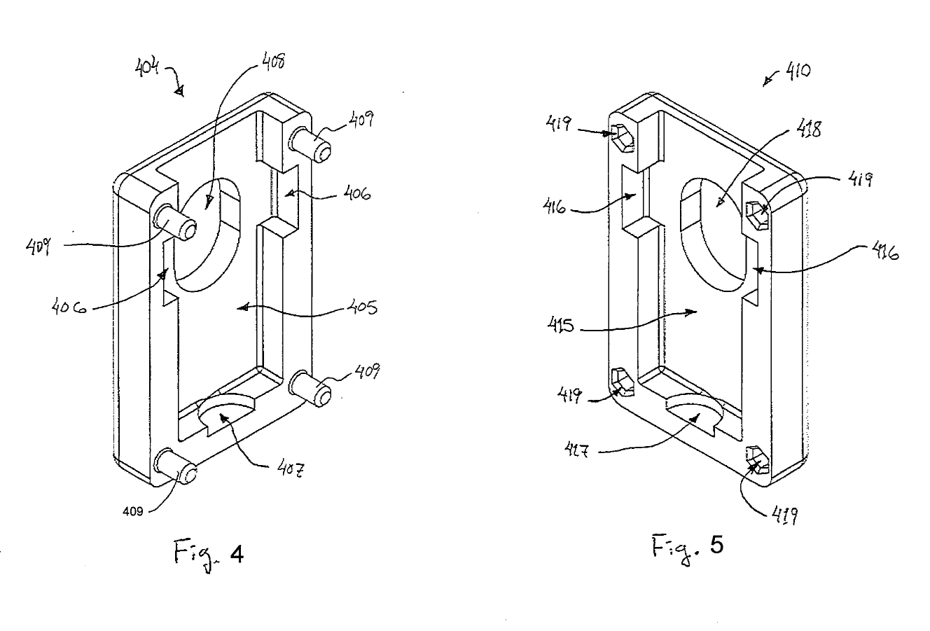

Distal member (404) is best seen in FIG. 4. As can be seen, distal member (404) comprises a generally rigid body defining a plurality of cavities (405, 406, 407) or other geometric features therein. For instance, distal member (404) defines a central cavity (405) laterally centered within distal member (404). As will be described in greater detail below, central cavity (404) is configured to receive at least a portion of locking assembly (420) to permit movement of locking assembly (420) therein.

Adjacent to central cavity (405), distal member (404) defines a pair of longitudinally aligned locating cavities (406). Each locating cavity (406) is on an opposite lateral side of central cavity (405), extending outwardly from central cavity (405) into distal member (404). As will be described in greater detail below, locating cavities (406) are generally configured to receive a corresponding portion of locking assembly (420) to guide locking assembly (420) along a predefined path as locking assembly (420) moves within central cavity (405). Although not shown, it should be understood that in some examples distal member (404) additionally defines certain guide features extending into locating cavities (406). In such examples, these guide features can further complement the guiding functionality of locating cavities (406).

Adjacent to the bottom of central cavity (405), distal member (404) defines a spring recess (407). Spring recess (407) comprises a generally semi-circular shape that is configured to receive resilient member (440). As will be described in greater detail below, spring recess (407) is generally configured to align resilient member (440) with locking assembly (420) within central cavity (405). This feature permits resilient member (440) to resiliently bias locking assembly (420) longitudinally while remaining in a fixed lateral position.

Distal member (404) further defines an opening (408) extending through distal member (404). Opening (408) is generally sized to correspond to the shape of the cannula such that the cannula can be received therethough. As will be described in greater detail below, opening (408) generally is positioned relative to a corresponding opening (418) in proximal member (410) such that both openings (408, 418) align when distal member (404) and proximal member (410) are coupled. This configuration permits cannula to pass entirely through housing (402). Accordingly, it should be understood that due to this configuration, Z-stop device (395) can be positioned at a variety of locations along the longitudinal length of cannula.

A plurality of protrusions (409) extend proximally from distal member (404). As will be described in greater detail below, protrusions (409) are configured to engage corresponding features of proximal member (410) to align proximal member (410) relative to distal member (404) when distal member (404) and proximal member (410) are coupled together. Protrusions (409) of the present example are shown as having a generally cylindrical shape. However, it should be understood that in other examples protrusions (409) may take on a variety of alternative shapes such as cubic, hexagonal, triangular, oval-shaped, and the like.

Proximal member (410) is best seen in FIG. 5. Housing (402) comprises at least two housing members. The first housing member is proximal member (410). The second housing member is distal member (404). It should be understood that due to the configuration of housing (402), proximal member (410) is generally configured as a mirror image of distal member (404), thereby defining substantially similar components. For instance like with distal member (404) described above, proximal member (410) comprises a generally rigid body defining a plurality of cavities (415, 416, 417) or other geometric features therein. In particular, proximal member (410) defines a central cavity (415) laterally centered within proximal member (410). As will be described in greater detail below, central cavity (414) is configured to receive at least a portion of locking assembly (420) to permit movement of locking assembly (420) therein.

Adjacent to central cavity (415), proximal member (410) defines a pair of longitudinally aligned locating cavities (416). Each locating cavity (416) is on an opposite lateral side of central cavity (415), extending outwardly from central cavity (415) into proximal member (410). As will be described in greater detail below, locating cavities (416) are generally configured to receive a corresponding portion of locking assembly (420) to guide locking assembly (420) along a predefined path as locking assembly (420) moves within central cavity (415). Although not shown, it should be understood that in some examples proximal member (410) additionally defines certain guide features extending into locating cavities (416). In such examples, these guide features can further complement the guiding functionality of locating cavities (416).

Adjacent to the bottom of central cavity (415), proximal member (410) defines a spring recess (417). Spring recess (417) comprises a generally semi-circular shape that is configured to receive resilient member (440). As will be described in greater detail below, spring recess (417) is generally configured to align resilient member (440) with locking assembly (420) within central cavity (405). This feature permits resilient member (440) to resiliently bias locking assembly (420) longitudinally while remaining in a fixed lateral position.

Proximal member (410) further defines an opening (418) extending through proximal member (410). Opening (418) is generally sized to correspond to the shape of cannula such that cannula can be received therethrough. As was similarly described above with respect to opening (408), opening (418) generally is positioned relative to a corresponding opening (408) in distal member (404) such that both openings (408, 418) align when distal member (404) and proximal member (410) are coupled. This configuration permits cannula to pass entirely through housing (402). Accordingly, it should be understood that due to this configuration, Z-stop device (395) can be positioned in a variety of locations along the longitudinal length of cannula.

A plurality of retainers (419) extend into proximal member (410). As was similarly described above with respect to protrusions (409) of distal member (404), retainers (419) are configured to receive corresponding protrusions (409) of distal member (404) to align proximal member (410) relative to distal member (404) when distal member (404) and proximal member (410) are coupled together. Retainers (419) of the present example are shown as having a generally hexagonal shape. However, it should be understood that in other examples retainers (419) may take on a variety of alternative shapes such as cubic, cylindrical, triangular, oval-shaped, and the like. Additionally, it should be understood that, as shown in FIGS. 4 and 5, retainers (419) of proximal member (410) and protrusions (409) of distal member (404) need not necessarily have identically corresponding shapes. Further still, it should be understood that in some examples, protrusions (409) of distal member (404) can be oversized relative to the size of retainers (419) of proximal member (410) to provide a press or friction fit.

FIGS. 6-8 show locking assembly (420) in greater detail. Locking assembly (420) comprises a generally rectangular body (422) with a pair of locating members (430) projecting laterally outwardly from the sides of body (422). Generally, body (422) is configured to fit within central cavities (405, 415) defined by distal and proximal members (404, 410). Similarly, locating members (430) are configured to fit within corresponding locating cavities (406, 416) of distal and proximal members (404, 410). The configuration permits body (422) to translate along a generally linear path within distal and proximal members (404, 410), with locating members (430) preventing body (422) from deviating from this path. As will be described in greater detail below, this translation of body (422) permits locking assembly (420) to move relative to cannula to selectively lock and release cannula.

Each locating member (430) further defines a locating recess (432). Each locating recess (432) extends longitudinally from each locating member (430) from top to bottom. As described above, in some examples locating cavities (406, 416) may include certain locating features. In such examples, these locating features may correspond to locating recess (432) of each locating member (430). It should be understood, however, that each locating recess (432) is entirely optional and may be omitted in some examples.

Body (422) defines a single cannula opening (424) extending transversely thorough body (422) from the distal face of body (422) to the proximal face. As is best seen in FIG. 7, opening (424) comprises an upper portion (426) and a lower portion (428). Upper portion (426) is defined by a generally oval-shaped or circular cross-sectional shape. It should be understood that this shape is generally configured to correspond to the shape of a corresponding upper portion of cannula. Accordingly, upper portion (426) is configured to receive cannula.

Lower portion (428) of opening (424) is defined by a generally rectangular cross-sectional shape. It should be understood that the generally rectangular shape of lower portion (428) is configured to permit some travel of locking assembly (420) relative to cannula. In other words, lower portion (428) is configured to permit some movement of locking assembly (420) relative to cannula while cannula remains stationary. As will be described in greater detail below, this configuration permits a pair of lock members (434) to move relative to cannula to selectively engage and disengage cannula.

Body (422) further includes a pair of lock members (434) projecting into opening (424). Each lock member (434) comprises a generally triangular prism shape. The widest point of each lock member (434) projects into the bottom of lower portion (428) of opening (424). Each lock member (434) then extends upwardly within opening (424) while progressively reducing in size. An engagement edge (436) of each lock member (434) defines the furthest point within opening (424). As will be described in greater detail below, this configuration of each lock member (434) is generally configured to engage with cannula to unlock and lock cannula relative to Z-stop device (395) as locking assembly (420) is translated relative to cannula. Although each lock member (434) of the present example is shown as having a generally triangular shape (best seen in FIG. 12), it should be understood that in some examples other shapes may be used. Alternatively, in some examples each lock member (434) is combined into a single lock member (434) for instance in such an example, engagement edge (436) may curve around the lower portion of body (422), thereby connecting each lock member (434) into a single integral lock member (434).

Each lock member (434) is integral with body (422). Thus, to at least some extent, body (422) defines the material characteristics of each lock member (434). For instance, in some examples body (422) is comprised of a generally rigid non-flexible material. In such examples, each lock member (434) is correspondingly rigid and non-flexible. This configuration can result in each lock member (434) digging into the surface of cannula, or alternatively temporarily deforming the exterior surface of cannula. In other examples, body (422) is comprised of a generally rigid, yet flexible material. In such examples, each lock member (434) is thus correspondingly rigid yet flexible. This configuration can result in each lock member (434) being relatively resilient, thereby permitting some deformation of each lock member (434) when engaging cannula. In still other examples, each lock member (434) comprises an elastomeric material or alternatively includes an elastomeric coating that is configured to provide increased friction when engaging cannula. In yet other examples, body (422) is comprised of a material with non-homogeneous properties such that body (422) generally includes one or more of the properties described above while each lock member (434) includes a different combination of any one or more of the properties described above. In yet other examples, body (422) and lock members (434) comprise separate components such that each can have different material properties as will be apparent to those of ordinary skill in the art in view of the teachings herein.

To laterally locate resilient member (440) relative to body (422), body (422) includes a receiving protrusion (438) extending downwardly from body (422). Receiving protrusion (438) comprises a generally cylindrical shape that corresponds to the shape of resilient member (440). It should be understood that the generally cylindrical shape of receiving protrusion (438) is generally configured to receive resilient member (40) thereon to thereby locate resilient member (140) relative to body (422). Accordingly, it should be understood that in examples that use a resilient member (40) other than a coil spring, receiving protrusion (138) may be modified to accommodate such a resilient member (440). Various alternative configurations of receiving protrusion (438) will be apparent to those of ordinary skill in the art in view of the teachings herein.

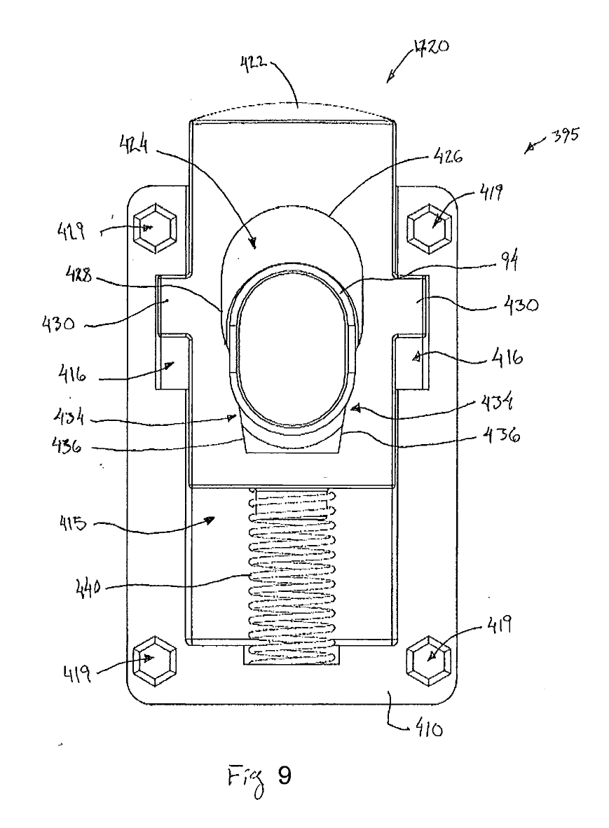

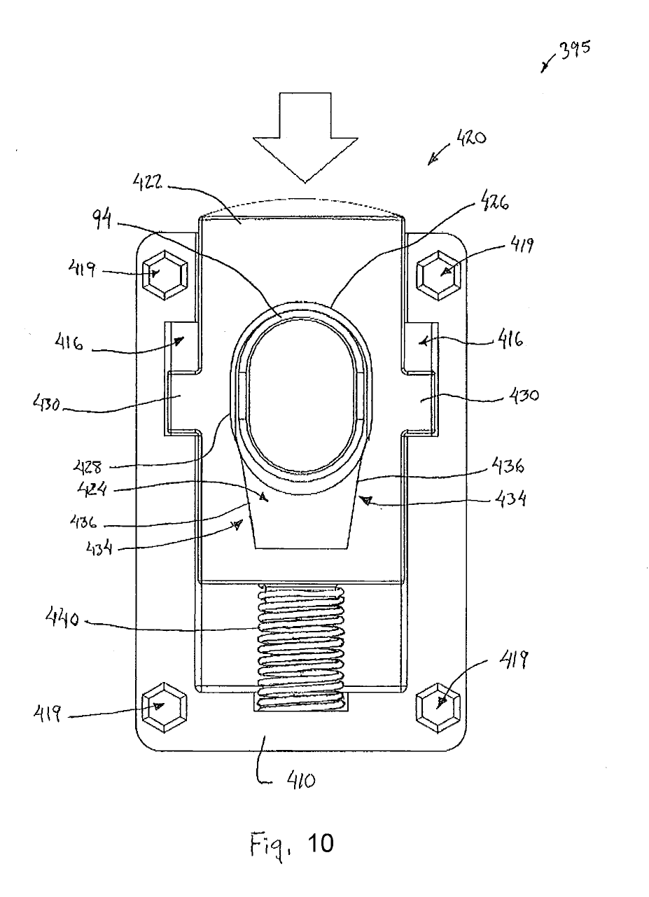

FIGS. 9, 10 and 11 show an exemplary use of Z-stop device (395). As is best seen in FIG. 9, Z-stop device (395) initially begins disposed on cannula in a locked configuration. Although Z-stop device (395) is shown and described herein as being initially disposed on cannula, it should be understood that in some examples Z-stop device (395) may be initially separated from cannula, thereby requiring an operator to first attach Z-stop device (395) to cannula. Regardless of whether cannula is initially disposed within Z-stop device (395) or separate from Z-stop device (395), it should be understood that Z-stop device (395) will initially be configured as shown in FIG. 9. A procedure for attaching Z-stop device 395 to cannula will be described in greater detail below.

In the initial position locked configuration, locking assembly (420) is biased upwardly by resilient member (440). In this position, locking assembly (420) is in its highest possible position. Further upward movement or translation of locking assembly (420) is prohibited by locating cavities (416), which terminate as shown to prevent further movement of locating members (430) of locking assembly (420).

With locking assembly (420) in the furthest upward position, lock members (434) of locking assembly (420) are pushed into engagement with cannula. It should be understood that cannula is prevented from moving upwardly and out of engagement with lock members (434) by openings (408, 418) of distal and proximal members (404, 410) of housing. As described above, openings (408, 418) are sized to correspond to the size and shape of cannula. It should be understood that in the present example openings (408, 418) are slightly oversized relative to the exterior of cannula to permit translation of cannula (when not locked) but prevent lateral movement of cannula.

With lock members (434) engaging cannula, lock members (434) generally generate friction between engagement edge (436) of each lock member (434) and cannula. In some examples this friction is the result of each lock member (434) digging into the outer surface of cannula. In other examples, this friction is the result of each lock member (434) resiliently deforming around cannula. In still other examples this friction is the result of some combination of both mechanisms. Regardless, the friction or other interference/obstruction with cannula generated by lock members (434) fixes the longitudinal position of cannula relative to locking assembly (420) and housing (402).

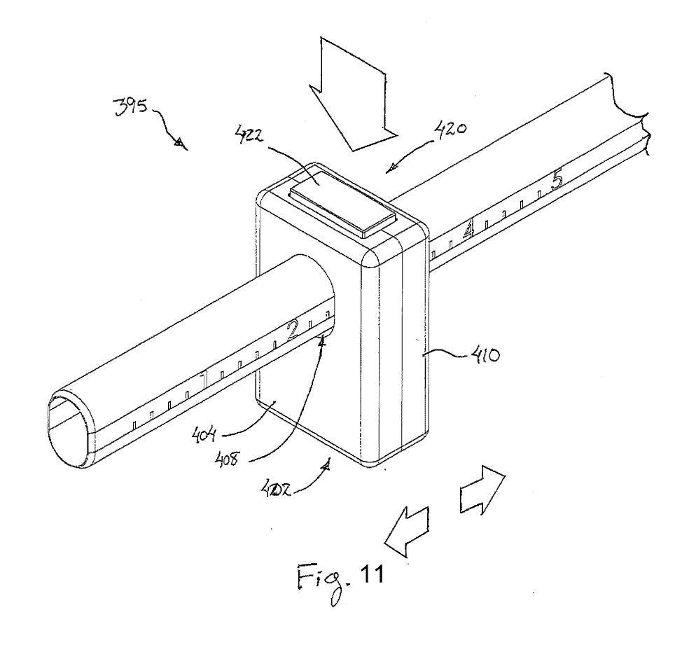

To release cannula, or to alternatively attach cannula in examples where cannula is not initially attached, an operator may push downwardly on the portion of body (422) that extends outwardly from housing (402). As can be seen in FIG. 10 this downward force against body (422) pushes locking assembly (420) downwardly against the resilient bias of resilient member (440) into an unlocked configuration. This downward motion can continue until locating members (430) of locking assembly (420) bottom out on the bottom of each respective locating cavity (416). As body (422) moves downwardly, lock members (430) are translated away from cannula until lock members (430) disengage from cannula.

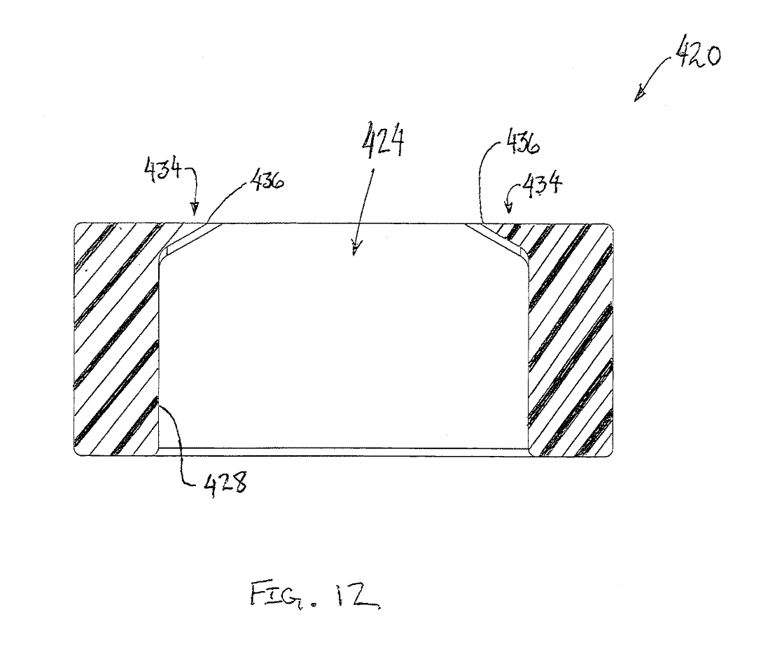

Once lock members (430) have been disengaged from cannula, cannula may be moved longitudinally as shown in FIG. 11. While body (422) is pushed downwardly to position locking assembly (420) in the unlocked configuration, an operator may reposition Z-stop device (395) along the length of cannula as desired. In some examples, this can involve moving Z-stop device (395) to correspond to a desired insertion depth for cannula. Alternatively, if Z-stop device (395) is initially separate from cannula, Z-stop device (395) can be inserted onto cannula at this point.

Once a desired position for Z-stop device (395) has been achieved along cannula, the operator may release body (422). Release of body (422) will cause resilient member (440) to return locking assembly (420) to the locked configuration as shown in FIG. 9. Accordingly, release of body (422) will result in locking Z-stop device (395) in the desired position along the length of cannula. An operator may then proceed with the targeting and biopsy procedure as described above. If at any point during the procedure an operator desires to alter the positioning of Z-stop device (395), the operator may do so by repeating the procedure described above with respect to FIGS. 9-11.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material,

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We Claim:

1. A device for use with a biopsy cannula, the device comprising:
(a) a housing defining a shaft aperture sized to receive and at least partially circumferentially encompass the biopsy cannula; and
(b) a locking assembly defining a lock opening configured to receive the biopsy cannula, wherein the locking assembly includes a lock member extending inwardly within the lock opening, wherein the lock member defines an engagement edge that is configured to bind against the biopsy cannula upon translation of the locking assembly relative to the shaft aperture to a locking position, wherein the locking assembly and housing are configured to restrict the depth of insertion of the biopsy cannula through the lock opening when the lock member of the locking assembly is bound against the biopsy cannula in the locking position.

2. The device of claim 1, further comprising a resilient member, wherein the resilient member is configured to resiliently bias the locking assembly towards the locking position.

3. The device of claim 2, wherein the resilient member is a coil spring.

4. The device of claim 1, wherein the locking assembly is translatable relative to the shaft aperture.

5. The device of claim 1, wherein the opening includes two housing members, wherein the two housing members are configured to contain at least a portion of the locking assembly.

6. The device of claim 5, wherein at least a portion of the locking assembly extends outwardly from the two housing members.

7. The device of claim 6, wherein the at least a portion of the locking assembly extending outwardly from the two housing members is configured to be pressed by an operator.

8. The device of claim 5, wherein the two housing members form a longitudinally split configuration.

9. The device of claim 1, wherein the lock opening is defined by a first portion and a second portion, wherein the first portion is configured to receive the biopsy cannula, wherein the second portion is configured to permit translation of the body of the locking assembly relative to the biopsy cannula

10. The device of claim 9, wherein the body of the locking assembly is translatable along a translation axis, wherein the translation axis is perpendicular to a longitudinal axis of the biopsy cannula.

11. The device of claim 9, wherein the lock member is associated with the second portion.

12. The device of claim 9, wherein the first portion includes a curved shape, wherein the second portion includes a rectangular shape.

13. The device of claim 1, wherein the lock member is generally rigid such that the lock member is configured to deform the biopsy cannula.

14. The device of claim 1, wherein the housing is configured to abut a guide cube associated with an MRI patient fixture to thereby cease axial movement of the housing relative to a patient.

15. The device of claim 1, wherein each of the shaft aperture and lock opening are

16. A targeting set for positioning a biopsy device under MRI guidance, the targeting set comprising:
(a) a sleeve assembly including a hub and a targeting sleeve extending distally from the hub, wherein the sleeve assembly is adapted to receive a needle of the biopsy device;
(b) an obturator, wherein the obturator is insertable within the targeting sleeve in lieu of the needle of the biopsy device; and
(c) a depth stop, wherein the depth stop includes:
(i) a housing, wherein the housing defines a sleeve opening sized to slidably receive the targeting sleeve of the sleeve assembly,
(ii) a resilient member, and
(iii) a locking assembly, wherein the locking assembly includes a body and at least one lock member, wherein the body includes a lock opening, wherein the lock opening is configured to receive the targeting sleeve of the sleeve assembly, wherein the at least one lock member extends inwardly from the body into the lock opening, wherein the resilient member is configured to translate the body relative to the housing to engage the at least one lock member with the targeting sleeve of the sleeve assembly to thereby selectively secure a longitudinal position of the targeting sleeve, wherein the at least one lock member is configured to secure the targeting sleeve bi-directionally along an axis defined by the targeting sleeve when the at least one lock member is engaged with the targeting sleeve.

17. The targeting set of claim 16, wherein the housing of the depth stop defines a pair of locating cavities, wherein the body of the locking assembly includes a pair of locating members, wherein the locating members are configured to engage the locating cavities of the housing to permit translation of the body along a single axis.

18. The targeting set of claim 16, wherein the at least one lock member is generally resilient such that the at least one lock member is configured to be deformable by the biopsy cannula.

19. The targeting set of claim 16, wherein the at least one lock member includes a complementary pair of lock members.

20. A method for aligning a device for use with a targeting cannula relative to the targeting cannula, the device including a housing, wherein the housing defines a cannula opening extending therethrough, a resilient member, and a lock body, wherein the lock body includes a lock opening and a pair of lock features extending inwardly into the lock opening, the method comprising the steps of:
(a) depressing the lock body relative to the housing against a resilient bias provided by the resilient member, wherein the step of depressing the lock body moves the pair of lock features away from the cannula opening of the housing;
(b) inserting the targeting cannula through the cannula opening of the housing and the lock opening of the lock body while continuing to depress the lock body;
(c) positioning the housing relative to the targeting cannula to align the housing with a location on the targeting cannula corresponding to a predetermined insertion depth; and
(d) releasing the lock body, wherein the step of releasing the lock body includes translating the lock body relative to the housing using the resilient member, wherein the step of releasing the lock body further includes driving the pair of lock features into engagement with the targeting cannula such that the pair of lock features bear against a generally uniform surface of the targeting cannula.
